(19) United States
(12) Patent Application Publication
(10) Pub. No.: US 2020/0126673 A1
(43) Pub. Date: Apr. 23, 2020

(54) EVALUATION REQUEST PROGRAM, EVALUATION REQUEST METHOD, AND COMPUTER APPARATUS

(71) Applicants: OMRON HEALTHCARE CO., LTD., Muko-shi (JP); OMRON Corporation, Kyoto-shi (JP)

(72) Inventors: Yuka TANABE, Kyoto (JP); Yu MURATA, Kyoto (JP)

(21) Appl. No.: 16/716,736

(22) Filed: Dec. 17, 2019

Related U.S. Application Data
(63) Continuation of application No. PCT/JP2018/028830, filed on Aug. 1, 2018.

(30) Foreign Application Priority Data
Aug. 9, 2017 (JP) 2017-153752

(51) Int. Cl. G16H 50/30; G16H 10/60; G16H 40/63; A61B 5/02

(52) U.S. Cl. CPC G16H 50/30; A61B 5/02; G16H 40/63; G16H 10/60

(57) ABSTRACT

A non-transitory computer readable medium storing a program which is executed by a computer to provide the steps of: communicating with a measurement device configured to perform a measurement on a body of an user to provide services, and operating an evaluation-target application that collects biological data indicating a result of the measurement as first data; determining whether or not content of the first data collected through an operation of the evaluation-target application matches an evaluation condition defining whether or not the data is preferable to a user; and causing a display to display a screen for requesting the user to input a review of the evaluation-target application if content of the first data matches the evaluation condition.

### CROSS-REFERENCE TO RELATED APPLICATIONS

This application is a Continuation Application of PCT Application No. PCT/JP2018/028830, filed Aug. 1, 2018 and based upon and claiming the benefit of priority from Japanese Patent Application No. 2017-153752, filed Aug. 9, 2017, the entire contents of all of which are incorporated herein by reference.

### FIELD

The present embodiments relate generally to an evaluation request program, an evaluation request method, and a computer apparatus.

### BACKGROUND

When selecting content for use, in a situation where various types of content are distributed, a user sometimes refers to evaluations posted by other users of that content (see, for example, Jpn. Pat. Appln. KOKAI Publication No. 2009-020724). In such cases, higher ratings of content generally encourage the user to use the content. For this reason, in order to deliver a situation where target content is used by many users, it is preferable to gather more reviews with high ratings for the target content.

### SUMMARY

An evaluation request program according to one aspect of the present invention is a program causing a computer having a display device to execute: a first step of operating an evaluation-target application that collects predetermined data to provide services; a second step of determining whether or not content of the predetermined data, collected by the evaluation-target application through its operation, matches an evaluation condition which defines whether or not the data is preferable for a user; and a third step of causing the display device to display a screen for requesting the user to input a review of the evaluation-target application.

A person is less reluctant to spare effort and more likely to behave positively when in a good mood. For this reason, it can be expected that more reviews with high ratings can be collected for the content if users are requested to write a review when in a good mood.

According to the above structure, it is possible to request a user to write a review of an evaluation-target application at timing when predetermined data collected through operation of the target application is determined to be preferable to users. In other words, at the timing when a user is presumably put in a good mood through the use of the target application, it is possible to request the user to write a review of that target application.

Thus, according to the above structure, it is possible to request a user who highly evaluates a target application to write a review at an appropriate timing, thereby leading to the collection of more reviews with high ratings for the target application. A type of an evaluation-target application is not necessarily limited, and can be selected as appropriate in accordance with an embodiment. An evaluation-target application may be an application that collects biological data from a measurement device, such as a body weight and composition monitor, an activity monitor, a blood pressure manometer, a blood glucose level measuring instrument, a pedometer, or a thermometer, etc. An evaluation-target application also manages the collected biological data.

In the evaluation request program according to the above aspect: the evaluation-target application may communicate with a measurement device configured to perform measurement on a body of the user and collect biological data indicating a result of the measurement as the predetermined data; the evaluation condition may define that content of the collected predetermined data is preferable to the user if there are signs of improvement in a health condition of the user; in the second step, the computer may be caused to determine whether or not there are signs of improvement in a health condition of the user based on the collected biological data; and in the third step, the computer may be caused to display a screen for requesting the user to input a review of the evaluation-target application when it is determined that there are signs of improvement in the health condition of the user. According to the structure, it is possible to request a user whose health condition is improved through the use of a target application to write a review of the target application. It is thereby expected that more reviews with high ratings can be collected for the target application.

In the evaluation request program according to the above aspect, the biological data relates to at least one of blood pressure, body weight, body fat, visceral fat, physical age, skeletal muscle rate, number of steps, activity amount, blood glucose level, basal body temperature rhythm, and sleep condition, and in the second step, if there are signs of improvement in any of those indices, the computer is caused to determine that there are signs of improvement in the health condition of the user. According to the structure, it is possible to request a user whose health condition is improved through the use of a target application, in terms of at least one of blood pressure, body weight, body fat, visceral fat, physical age, skeletal muscle rate, number of steps, activity amount, blood glucose level, basal body temperature rhythm, or sleep condition, to write a review of the target application. It is thereby expected that more reviews with high ratings can be collected for the target application.

The evaluation request program according to the above aspect may cause the computer to further execute the following steps: a fourth step of monitoring an operation stream of the user when the evaluation-target application is used; a fifth step of determining whether or not the operation stream satisfies a predetermined condition; and a sixth step of displaying a screen for requesting the user to input a review of the evaluation-target application on the display. According to the structure, it is possible to determine whether or not the user uses the target application comfortably and free of stress, and to request the user who uses the target application comfortably and free of stress to write a review of the target application. It is thereby expected that more reviews with high ratings can be collected for the target application.

According to the evaluation request program according to the above aspect, in the fourth step, the computer may be caused to measure at least one of a length of time staying on an operation screen or an operation path, and in the fifth step, the computer may be caused to determine whether or not the operation stream satisfies a predetermined condition based on at least one of the length of time staying on the operation screen or the operation path. According to the structure, it is possible to determine whether or not the user uses the target application comfortably and free of stress based on at least one of the length of time staying on the operation screen or the operation path. Thus, it is possible to request a user who uses the target application comfortably and free of stress to write a review of the target application.

The evaluation-target application may cause the computer to communicate with a predetermined apparatus when collecting the predetermined data. In the fourth step, the evaluation request program according to the above aspect may cause the computer to measure a status of the communication with the predetermined apparatus as the operation stream, and in the fifth step, the program may cause the computer to determine whether or not the operation stream satisfies a predetermined condition based on the communication status. According to the structure, it is possible to determine whether or not the user uses the target application comfortably and free of stress based on the status of the communication with the predetermined apparatus. Thus, it is possible to request a user who uses the target application comfortably and free of stress to write a review of the target application.

As another example of the evaluation request program according to the foregoing aspect, an information processing apparatus that realizes each of the foregoing structures, an information processing method, or a storage medium storing an evaluation request program according to the foregoing aspect thereon and readable by a computer or other apparatus or machine is provided. Herein, the storage medium readable by a computer, etc. is a medium storing information of programs, etc. in an electronical, magnetic, optical, mechanical, or chemical manner.

For example, the evaluation request method according to one aspect of the present invention is an information processing method in which a computer having a display device executes the following steps: a first step of operating an evaluation-target application that collects predetermined data to provide services; a second step of determining whether or not content of the predetermined data collected by the operation of the evaluation-target application matches an evaluation condition defining whether or not the content is preferable for a user; and a third step of causing the display device to display a screen for requesting the user to input a review of the evaluation-target application when the content of the collected predetermined data matches the evaluation condition.

In the evaluation request method according to the above aspect: the evaluation-target application may collect biological data indicating a result of the measurement as the predetermined data through a communication with a measurement device configured to perform measurement on a body of the user; the evaluation condition may define that content of the collected predetermined data is preferable to the user if there are signs of improvement in a health condition of the user; in the second step, the computer may be caused to determine whether or not there are signs of improvement in a health condition of the user based on the collected biological data; and in the third step, the computer may be caused to display a request for the user to input a review of the evaluation-target application when it is determined that there are signs of improvement in the health condition of the user.

For example, the computer apparatus according to one aspect of the present invention includes: one or more processors; a memory storing an evaluation-target application and a program in such a manner that they are executable by the one or more processors; and a display device, and the one or more processors are an information processing apparatus that executes the following steps in accordance with the program: a first step of operating an evaluation-target application that collects predetermined data to provide services; a second step of determining whether or not content of the predetermined data collected by the operation of the evaluation-target application matches an evaluation condition defining whether or not the content is preferable for a user; and a third step of causing the display device to display a screen for requesting the user to input a review of the evaluation-target application when the content of the collected predetermined data matches the evaluation condition.

In the computer apparatus according to the above aspect: the evaluation-target application may communicate with a measurement device configured to perform measurement on a body of the user, and collect biological data indicating a result of the measurement as the predetermined data; the evaluation condition may define that content of the collected predetermined data is preferable to the user if there are signs of improvement in a health condition of the user; in the second step, the one or more processors may determine whether or not there are signs of improvement in a health condition of the user based on the collected biological data; and in the third step, the one or more processors may display a screen for requesting the user to input a review of the evaluation-target application when it is determined that there are signs of improvement in the health condition of the user.

According to the present invention, it is possible to provide a technique of requesting a user who has an evaluation with high rating for a target application to write a review at appropriate timing.

### BRIEF DESCRIPTION OF THE DRAWINGS

FIG. 1 schematically shows an example of a situation to which the present invention is applied.

FIG. 2 schematically shows an example of a hardware configuration of a computer apparatus according to an embodiment.

FIG. 3 schematically shows an example of a hardware configuration of a measurement device according to the embodiment.

FIG. 4 schematically shows an example of a software configuration of the computer apparatus according to the embodiment.

FIG. 5 schematically shows an example of a procedure of the processing in the computer apparatus according to the embodiment.

FIG. 6 shows an example of an application screen.

FIG. 7 shows an example of the application screen.

FIG. 8 shows an example of a screen for requesting a user to post a review.

FIG. 9 shows an example of a screen for accepting input of reviews.

### DETAILED DESCRIPTION

It is presumed that a person who uses target content with a high frequency will potentially offer a good evaluation of that target content. Therefore, as a method of collecting evaluations with high ratings, a method of requesting a user who uses the target content with a high frequency to write a review is possible. However, it takes time and effort for a user to write and post a review. Until now, when referring only to an index of frequency of use, it has been difficult to predict with certainty whether a user would expend time and effort upon posting a review in response to a request to do so.

In one aspect, the present embodiments are proposed in consideration of such a circumstance, and an object of the present embodiments are to provide a technique of requesting a user who evaluates a target application highly to write a review at appropriate timing.

To solve the above-described problem, the present embodiments adopt the following structures.

In the following, an embodiment of one aspect of the present invention (may be referred to as “the present embodiment” hereinafter) will be described with reference to the drawings. It should be noted, however, that the embodiment described hereinafter is merely an example of the present embodiment in every respect. As a matter of course, various alternations and modifications can be made, without departing from the gist of the embodiment. In other words, to realize the present embodiment, a specific structure may be adopted as appropriate in accordance with an embodiment. Data described in the present embodiment is explained in the form of natural language; however, in a real situation, the data is designated by a pseudo language, commands, parameters, or machine words, etc. recognizable for computers.

### 1. EXAMPLE OF APPLICATION

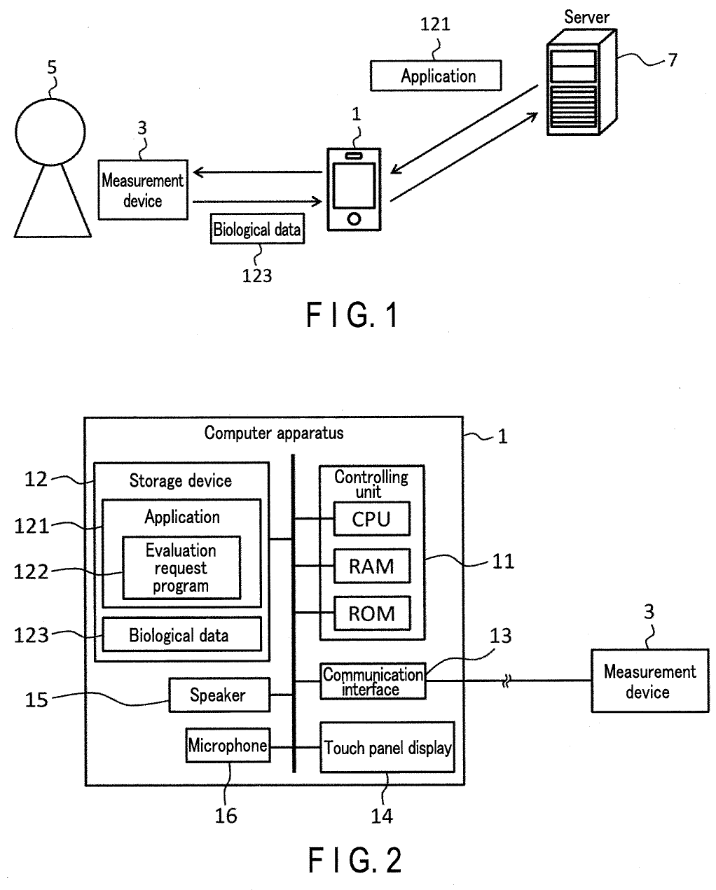

First, an example of an application of the present embodiment will be described with reference to FIG. 1. FIG. 1 schematically shows an example of an application of a computer apparatus 1 according to the present embodiment.

As shown in FIG. 1, an application 121 provided from an external server 7 is installed onto the computer apparatus 1 according to the present embodiment. A user 5 operates the computer apparatus 1 to activate the application 121 and receive provision of predetermined services. The application 121 corresponds to “evaluation-target application” in the present embodiment.

The computer apparatus 1 has a display device (later-described touch panel display 14), and operates the application 121. The application 121 collects predetermined data to provide services. The computer apparatus 1 determines whether or not content of the collected predetermined data matches an evaluation condition defining whether or not the data is preferable to the user 5. Then, if the content of the collected predetermined data matches the evaluation condition, the computer apparatus 1 causes the display device to display a screen for requesting the user 5 to post a review of the application 121.

The application 121 is not particularly limited, as long as it obtains some kind of data when services are provided. The evaluation condition may be determined as appropriate in accordance with a type of collected data. As an example, in the present embodiment, an application that cooperates with the measurement device 3 configured to perform measurement on the body of the user 5 is utilized as the application 121. The evaluation condition is defined so as to determine that content of the collected predetermined data is preferable to the user 5 if there are signs of improvement in the health condition of the user 5.

Specifically, the measurement device 3 is, for example, a body weight and composition monitor, a blood pressure manometer, an activity monitor, a blood glucose level measuring instrument, a pedometer, a thermometer, a sleep measuring instrument, a heart rate monitor, or an electro cardiograph, etc. The application 121 collects biological data, which is a measurement result obtained by the measurement device 3, and manages the collected biological data, so as to provide a service of displaying history of living body information of the user obtained by the measurement device 3 in a form of graph, etc. In other words, the application 121 collects the biological data 123 indicating a measurement result as predetermined data through communication with the measurement device 3.

Herein, the computer apparatus 1 according to the present embodiment determines whether or not there are signs of improvement in the health condition of the user 5, based on the collected biological data 123. Then, if the content of the collected predetermined data matches the evaluation condition, the computer apparatus 1 causes the display device to display a screen for requesting the user 5 to post a review of the application 121. The review input by the user 5 is transmitted to an external server 7 via a network, etc., for example. Data of reviews posted by each user is thus accumulated in the external server 7.

As described above, in the present embodiment, it is possible to request a user to write a review of the application 121 at a timing when predetermined data collected through an operation of the application 121 is determined to be preferable to the user 5. In the present embodiment, it is possible to request the user 5 to write a review of the application 121 at a timing when the health condition of the user 5 is improved through use of the application 121.

Herein, it is inferred that a person would be less reluctant to spare effort and more likely to behave positively when in a good mood. In the present embodiment, it is possible to request the user 5 to write a review of the application 121 at a timing when the mood of the user 5 is improved as a result of improvement of their health condition, and when the user 5 has a good impression of the application 121. Thus, according to the present embodiment, it is possible to request the user, who highly values the application 121, to write a review at an appropriate timing, and thereby possible to collect more reviews with high ratings for the application 121.

### 2. EXAMPLES OF CONFIGURATION

[Hardware Configuration]

<Computer Apparatus>

Next, an example of a hardware configuration of the computer apparatus 1 according to the present embodiment will be described, with reference to FIG. 2. Next, an example of a hardware configuration of the computer apparatus 1 according to the present embodiment will be described, with reference to FIG. 2.

As shown in FIG. 2, the computer apparatus 1 according to the present embodiment is a computer in which the controlling unit 11, the storage device 12, the communication interface 13, the touch panel display 14, the speaker 15, and the microphone 16 are electrically connected to each other. In FIG. 2, the communication interface is shown as “communication I/F”.

The controlling unit 11 includes a central processing unit (CPU), a random access memory (RAM), and a read only memory (ROM), etc., which serve as hardware processors, and controls each constituent element in accordance with information processing. The CPU corresponds to the “processor” of the present embodiment. The storage device 12 is comprised of, for example, a hard disk drive, a solid state drive, an optical disk, a magnetic disk, a flash memory, or a memory card, etc., and stores various types of data, such as the application 121, the evaluation request program 122, and the biological data 123. The storage device 12 corresponds to the “memory” of the present embodiment.

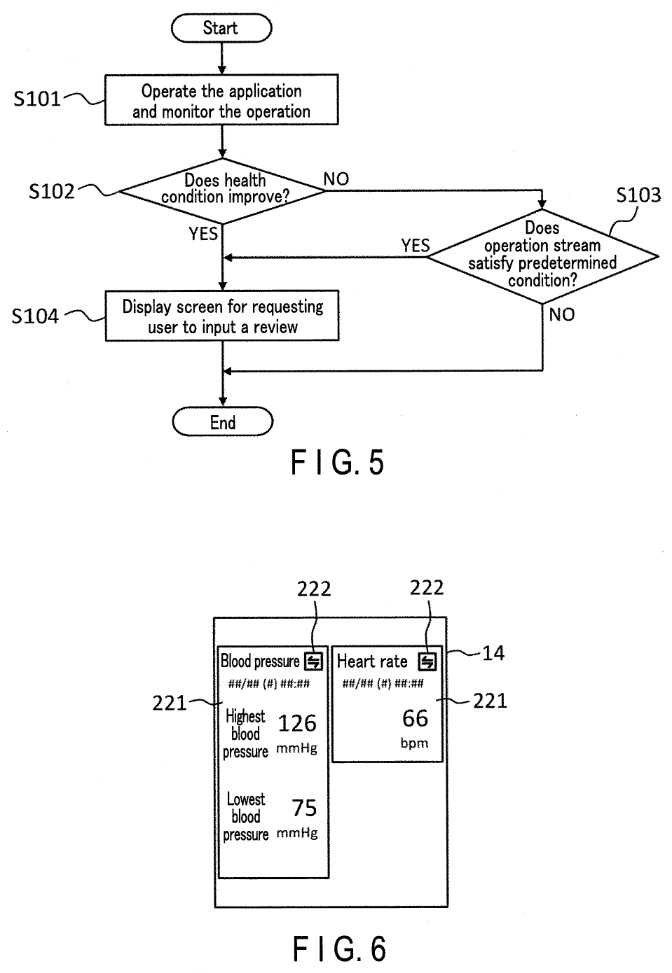

The application 121 collects the biological data 123 from the measurement device 3 as described above, and is a software for managing the collected biological data 123. The biological data 123 is collected by operating the application 121 after a communication between the computer apparatus 1 and the measurement device 3 is established. The evaluation request program 122 is a program for causing the computer apparatus 1 to perform the later-described information processing to request the user 5 to post a review of the application 121 (FIG. 5). In the present embodiment, the evaluation request program 122 is incorporated in the application 121. However, an aspect of provision of the evaluation request program 122 is not limited to such an example, and the evaluation request program 122 may be provided separately from the application 121. The details will be described later.

The communication interface 13 is, for example, a wired local area network (LAN) module, or a wireless LAN module, etc., and is an interface for wired or wireless communication via a network. The type of the communication interface 13 may be selected as appropriate in accordance with a target of connection (for example, the measurement device 3 or the server 7). The communication interface 13 may be configured as appropriate in accordance with a type of a communication standard. In the present embodiment, the computer apparatus 1 is connected to the measurement device 3 and the server 7 via the communication interface 13. Bluetooth (trademark) may be adopted for a communication standard between the computer apparatus 1 and the measurement device 3.

Any publicly-known touch panel display may be used as the touch panel display 14, and for inputting a message and displaying an image, etc. The touch panel display 14 is an example of the “display device” of the present embodiment. The user 5 can operate the computer apparatus 1 via the touch panel display 14. The speaker 15 and the microphone 16 may be a publicly-known speaker and microphone, and they are used for audio input and output.

Omission, replacement, or addition of a constituent element can be made in the specific hardware configuration of the computer apparatus 1 as appropriate, in accordance with an actual situation where the present embodiment is realized. For example, the controlling unit 11 may include a plurality of hardware processors. Each hardware processor may be comprised of a microprocessor or a field-programmable gate array (FPGA), etc. The computer apparatus 1 may be an information processing apparatus designed specifically for providing a service, or a mobile device including a desktop personal computer (PC), a tablet PC, or a smart phone.

The computer apparatus 1 may be connected to a drive apparatus, etc. for reading data stored in the storage device. In this case, the application 121 may be provided through a storage medium. If a drive apparatus is connected to the computer apparatus 1, the application 121 including the evaluation request program 122 may be stored in the storage medium. The storage medium is a medium storing information of programs recorded thereon in an electronical, magnetic, optical, mechanical, or chemical manner so that the information of programs is readable for a computer, device or machine. The storage medium is, for example, a compact disk (CD), a digital versatile disk (DVD), or a flash memory, etc.

<Measurement Device>

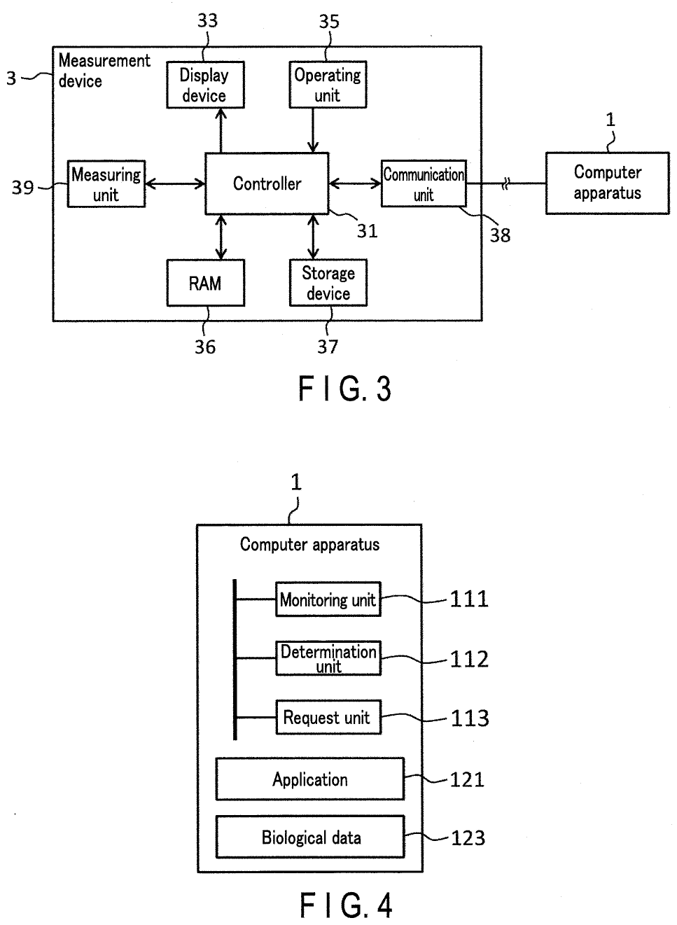

Next, an example of a hardware configuration of the measurement device 3 according to the present embodiment will be described with reference to FIG. 3. FIG. 3 schematically shows an example of a hardware configuration of the measurement device 3 according to the present embodiment. The measurement device 3 is configured to perform measurement on the body of the user 5 as appropriate.

As shown in FIG. 3, the measurement device 3 of the present embodiment is a computer having a controller 31, and a display 33, an operating unit 35, a RAM 36, a storage device 37, a communication unit 38, and a measuring unit 39 respectively connected to the controller 31. The measurement device 3 is, for example, a body weight and composition monitor, a blood pressure manometer, an activity monitor, a blood glucose level measuring instrument, a pedometer, a thermometer, a sleep measuring instrument, a heart rate monitor, or an electro cardiograph, etc., and may be configured as appropriate in accordance with a type of information to be measured. The measurement device 3 is an example of “predetermined apparatus” of the present embodiment.

The controller 31 is configured to control the operation of each unit through a microcomputer or an FPGA (field-programmable gate array) etc., for example. The display 33 is configured to display various types of information, on a liquid crystal display or an organic EL display, for example. The operating unit 35 is configured to accept a user operation input via the buttons or the touch panel, for example. The operating unit 35 may be comprised of buttons physically provided in the measurement device 3. If the touch panel display is used as the display 33, the operating unit 35 may be comprised of virtual buttons displayed on the display 33.

The RAM 36 may be a DRAM or an SRAM, etc. It temporarily stores data and is used as a working storage area for the controller 31. The storage device 37 is comprised of, for example, a hard disk drive, a solid state drive, an optical disk, a magnetic disk, a flash memory, or a memory card, etc., and stores biological body data (for example, various measurement values and index values calculated from the measurement values).

The communication unit 38 is the same as the communication interface 13, and is, for example, a wired LAN module or a wireless LAN module, etc. The measurement device 3 is connected to the computer apparatus 1 via the communication unit 38. In the present embodiment, the communication unit 38 is a module for performing communication in accordance with Bluetooth (trademark), for example.

The measuring unit 39 may be configured as appropriate in accordance with a target of measurement. For example, the measuring unit 39 may be comprised of a sensor that measures various types of information, such as blood pressure, heart rate, body weight, body fat, visceral fat, number of steps, activity amount, blood glucose level, body temperature, and sleep condition, etc. When a current location of the measurement device 3 is measured, the measuring unit 39 may have a receive circuit for receiving a global positioning system (GPS) signal, or the like. The measurement device 3 configured to measure a current location is also capable of measuring a walking distance, etc. of the user 5.

The controller 31 stores data, such as blood pressure, heart rate, body weight, body fat, visceral fat, number of steps, activity amount, body temperature, and sleep condition, etc. obtained by the measuring unit 39 in the storage device 37, as the biological body data. The controller 31 calculates various information items, such as physical age, skeletal muscle rate, body mass index (BMI), and basal body temperature rhythm, etc., based on measurement values obtained through the measurement, and causes the storage device 37 to store the calculated information as biological body data.

The sleep condition can be expressed by sleeping hours and waking hours, for example. The sleeping hours and waking hours can be measured by capturing weak radio waves caused by body motions (chest motion, a rollover in bed, etc.) with the measuring unit 39 during sleep, and determining whether or not the user 5 is asleep or awake. The sleep condition may be expressed by a length of time between going to bed and falling asleep, sleeping hours, or a length of time taken by the user to wake up, for example.

The skeletal muscle rate can be calculated based on set values, such as body height, age, impedance, and body weight, etc., and measurement values thereof. The basal metabolic rate can be calculated based on this skeletal muscle rate. The physical age can be calculated based on this basal metabolic rate. The relationship between the skeletal muscle and the basal metabolic rate, and the relationship between the basal metabolic rate and the physical age may be given by a predetermined equation or a correspondence table. Furthermore, the basal body temperature rhythm can be expressed by the relationship between menstrual period, ovulation day, and history of body temperatures.

As an example of the above-described communicable measurement device 3, the blood pressure manometers (HEM-7281T, HEM-7271T, etc.), the body weight and composition monitors (HBF-255T, etc.), and the activity monitors (HJA-405T, etc.), manufactured by OMRON corporation, can be given.

Similar to the computer apparatus 1, omission, replacement, or addition of a constituent element can be made in the specific hardware configuration of the measurement device 3 as appropriate, in accordance with an actual situation where the present embodiment is realized. In the measurement device 3 according to the present embodiment, the part for measuring information relating to the body of the user 5 (measuring unit 39) and the part for performing various information processing (controller 31, RAM 36, storage device 37, and communication unit 38) are integrated. However, the configuration of the measurement device 3 is not limited to such an example. For example, the measurement device 3 may be configured by using a general-purpose PC for the part for performing various information processing, and connecting an apparatus for measuring information relating to the body of the user 5 to the general-purpose PC.

<Others>

The server 7 can be configured with a computer in which a controller, a storage unit, a communication interface, an input unit, and an output unit are electrically connected to each other, similarly to the computer apparatus 1. In the storage unit, data of applications to be provided and data of reviews of the applications are stored. The server 7 is configured to provide, through the communication interface, the applications and the reviews thereof to a client device, such as the computer apparatus 1, etc., via the network. The server 7 is further configured to receive data of posted reviews from a client device, such as the computer apparatus 1, etc., and store the received posted data in a database. For this server 7, a publicly-known server apparatus, such as a server constituting a cloud, can be adopted. The server 7 may be comprised of one or more computers.

[Software Configuration]

Next, an example of a software configuration of the computer apparatus 1 according to the present embodiment will be described, with reference to FIG. 4. FIG. 4 schematically shows an example of a software configuration of the computer apparatus 1 according to the present embodiment.

The controlling unit 11 of the computer apparatus 1 expands the evaluation request program 122 stored in the storage device 12 in the RAM. Then, the controlling unit 11 interprets and executes, courtesy of the CPU, the evaluation request program 122 expanded in the RAM to control each constituent element. Thus, as shown in FIG. 4, the computer apparatus 1 according to the present embodiment is configured as a computer comprising a monitoring unit 111, a determination unit 112, and a request unit 113 as software modules.

The monitoring unit 111 monitors the operation of the evaluation-target application 121 that collects predetermined data to provide services. In the present embodiment, the monitoring unit 111 monitors an operation stream of the user 5 when the application 121 is used.

When the application 121 operates, in other words, the user 5 uses the application 121, the determination unit 112 determines whether or not content of the collected predetermined data matches an evaluation criterion defining whether or not the data is preferable to the user 5. Then, if the content of the collected predetermined data indeed matches the evaluation condition, the request unit 113 requests the user 5 to write a review of the application 121.

As described above, in the present embodiment, the application 121 collects the biological data 123 indicating a result of measurement performed by the measurement device 3 as predetermined data, through communication with the measurement device 3. The evaluation condition is defined so as to determine that content of the collected predetermined data is preferable to the user 5 if there are signs of improvement in the health condition of the user 5.

Herein, the determination unit 112 according to the present embodiment determines whether or not there are signs of improvement in the health condition of the user 5, based on the collected biological data 123. Then, if it is determined that there are signs of improvement in the health condition of the user 5, the request unit 113 requests the user 5 to write a review of the application 121.

In the present embodiment, in addition to the above situation where signs of improvement are found in the health condition, the computer apparatus 1 requests the user 5 to write a review of the application 121 in the case where the operation stream of the user 5 satisfies a predetermined condition. In other words, the determination unit 112 determines whether or not the operation stream of the user 5 satisfies the predetermined condition. Then, if it is determined that the operation stream of the user 5 indeed satisfies the predetermined condition, the request unit 113 requests the user 5 to write a review of the application 121.

The operation of each software module of the computer apparatus 1 will be described in detail in the later-described example of operation. In the present embodiment, an example where each software module of the computer apparatus 1 is realized by a general-purpose CPU is described. However, a part or all of the software modules may be realized by one or more dedicated processors. Furthermore, omission, replacement, or addition of a software module can be made in the software configuration of the computer apparatus 1 as appropriate, in accordance with an actual situation where the present embodiment is realized.

### 3. EXAMPLE OF OPERATION

Next, an operation example of the computer apparatus 1 will be described with reference to FIG. 5. FIG. 5 is a flow chart of an example of a procedure of the processing in the computer apparatus 1 according to the present embodiment. The processing procedure for requesting a review, which will be described below, corresponds to the “evaluation request method” of the present embodiment. The processing procedure described hereinafter is merely an example, and the procedure can be changed to the degree that such changes are possible. Omission, replacement, or addition of a step in the processing procedure described hereinafter can be made as appropriate, in accordance with an actual situation where the present embodiment is realized.

(Step S101)

In step S101, the controlling unit 11 operates the application 121. The controlling unit 11 functions as a monitoring unit 111, and monitors the operation of the application 121, namely the operation of the application 121 by the user 5.

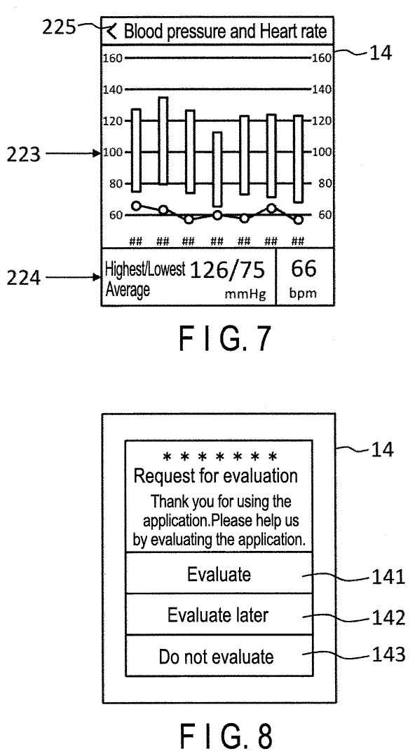

Herein, an example of the operation of the application 121 is described with reference to FIGS. 6 and 7. FIGS. 6 and 7 schematically show examples of screens of the application 121. When the user 5 performs measurement of information relating to their body using the measurement device 3, a result of the measurement is stored in at least one of the RAM 36 or the storage device 37 of the measurement device 3 as biological data 123. Thereafter, the user 5 operates the computer apparatus 1 to activate the application 121 and establish a communication between the computer apparatus 1 and the measurement device 3, and the computer apparatus 1 obtains biological data 123 from the measurement device 3, as an operation of the application 121. By repeating this process, the biological data 123 of the user 5 is collected in the computer apparatus 1. The controlling unit 11 displays the collected biological data 123 on the touch panel display 14 as a screen of the application 121.

FIG. 6 shows an example of an operation screen indicating the latest biological data 123 displayed on the touch panel display 14. This screen example includes panels 221 indicating measurement results of a blood pressure and a heart rate, and update buttons 222 for obtaining the measurement results once again from the measurement device 3 through a communication with the measurement device 3. The user 5 activates the application 121 by tapping the update button 222 or dragging down the whole screen, and operates the computer apparatus 1 so as to obtain the latest biological data 123 from the measurement device 3.

The user 5 can cause the touch panel display 14 of the computer apparatus 1 to display a history of each measurement result shown in FIG. 7, by tapping each panel 221. The screen example shown in FIG. 7 includes a graph displaying area 223, an average value displaying area 224, and a return button 225.

In the graph displaying area 223, the history of the measurement results is displayed in the form of a graph. The average value displaying area 224 displays an average value of each measurement result in accordance with a display format of the graph displaying area 223. In the example shown in FIG. 7, in the graph displaying area 223, the values of blood pressure and heart rate for a week are displayed in the form of graph; in the average value displaying area 224, they are displayed as numbers. The user 5 can return the display of the touch panel display 14 from the screen of FIG. 7 to the screen of FIG. 6 by tapping the return button 225.

The controlling unit 11 monitors the operation of the application 121 that exhibits such screen transitions so as to measure the operation stream of the user 5 when the application 121 is used. For example, the controlling unit 11 measures at least one of a length of time staying on each operation screen or an operation path as an operation stream. Thus, the operation stream of the user can be expressed with at least one of the length of time staying on each operation screen or the operation path. The controlling unit 11 may have a timer (not shown), and may measure the length of time staying on each screen with the use of the timer. The controlling unit 11 may measure a status of communication with the measurement device 3 as an operation stream. Thus, the operation stream of the user can be expressed through the status of communication with the measurement device 3. When the operation stream is measured through monitoring, the controlling unit 11 stores operation history information indicating the measured operation stream of the user in at least one of the RAM or the storage device 12.

In the example shown in FIG. 7, the measurement results for a week are displayed in the graph displaying area 223. However, the display format of the graph displaying area 223 is not limited to such an example. For example, the controlling unit 11 may display graphs in day, week, month, and year in the graph displaying area 223. The display formats for day, week, month, and year may be switchable in accordance with a user's operation. In this case, in response to the switching of the display format for the graph displaying area 223 upon the operation of the user 5, the controlling unit 11 displays each average value corresponding to the designated display format in the average value displaying area 224.

(Step S102)

In step S102, the controlling unit 11 functions as the determination unit 112, and determines whether or not there are signs of improvement in the health condition of the user 5, based on the collected biological data 123. If it is determined that there are signs of improvement in the health condition of the user 5, the controlling unit 11 advances the processing to step S104. On the other hand, if it is determined that there are no signs of improvement in the health condition of the user 5, the controlling unit 11 advances the processing to step S103.

A method of determining whether or not there are signs of improvement in the health condition of the user 5, based on the collected biological data 123 may be determined as appropriate in accordance with an embodiment. For example, a condition indicating improvement of the health condition is set for the collected biological data 123. The condition is, for example, incorporated into the evaluation request program 122, and stored in the storage device 12. In this case, the controlling unit 11 determines whether or not the collected biological data 123 matches the condition to determine whether or not there are signs of improvement in the health condition of the user 5.

In the present embodiment, the collected biological data 123 is related to at least one of, for example, blood pressure, body weight, body fat, visceral fat, physical age, skeletal muscle rate, number of steps, activity amount, blood glucose level, basal body temperature rhythm, or sleep condition. For this reason, the controlling unit 11 refers to the collected biological data 123, and determines whether or not there are signs of improvement in at least one of blood pressure, body weight, body fat, visceral fat, physical age, skeletal muscle rate, number of steps, activity amount, blood glucose level, basal body temperature rhythm, or sleep condition.

For example, regarding body weight, the controlling unit 11 can determine that there are signs of improvement if a recent measurement value becomes closer to an ideal value than a past measurement value did. Specifically, the controlling unit 11 determines whether or not the recent measurement value is closer to an ideal value than the past measurement value was. The controlling unit 11 expresses a change from the past measurement value to the recent measurement value with at least one of a rate or an amount of change, and determines whether or not at least one of the rate or the amount of change is greater than a threshold.

Then, if the recent measurement value is closer to an ideal value than the past measurement value was, and at least one of the rate or the amount of change is greater than a threshold, the controlling unit 11 can determine that there are signs of improvement regarding body weight. If not, the controlling unit 11 can determine that there are no signs of improvement regarding body weight.

The ideal value and the threshold value may be set as appropriate in accordance with a provider, etc. of the application 121. The ideal value and the threshold are stored in the storage device 12, for example. As for blood pressure, body weight, body fat, visceral fat, physical age, skeletal muscle rate, number of steps, activity amount, blood glucose level, whether or not there are signs of improvement can be determined by a method similar to the method adopted for body weight.

For example, regarding the basal body temperature rhythm, the controlling unit 11 determines that there are signs of improvement when the history of body temperature indicates a higher temperature period and a lower temperature period, and a menstrual cycle becomes almost constant, and this menstrual cycle falls within a predetermined range of values.

For example, as for sleep condition, the controlling unit 11 can determine that there are signs of improvement when a body motion during sleep drops, interrupted sleep drops, waking before a scheduled wake-up time drops, or a time taken until the user falls asleep drops. For a user with sleeping hours shorter than a predetermined length of time, the controlling unit 11 can determine that there are signs of improvement in the sleep condition when the sleeping hours of this user increase.

(Step S103)

In the next step S103, the controlling unit 11 functions as the determination unit 112, and determines whether or not the operation stream of the user 5 specified in step S101 satisfies a predetermined condition. If it is determined that the operation stream of the user 5 indeed satisfies a predetermined condition, the controlling unit 11 proceeds the processing to step S104. In contrast, if it is determined that the operation stream of the user 5 does not satisfy a predetermined condition, the controlling unit 11 omits the processing in step S104, and finishes the processing according to the present operation example.

The predetermined condition can be set as appropriate in accordance with an embodiment, so as to determine that the user 5 has a good impression about the application 121. For example, the predetermined condition may be set so as to determine whether or not the user 5 can use the application 121 comfortably and free of stress.

Whether or not the application 121 is used comfortably and free of stress can be determined based on at least one of, for example, the length of staying time on each operation screen or the operation path measured in step S101. As a specific example, the controlling unit 11 may determine that the operation stream satisfies a predetermined condition if a length of staying time on each operation screen during the operation flow is shorter than a threshold, or the number of returns to the operation screen is equal to or lower than a threshold. The threshold is stored in the storage device 12, for example.

It is also possible to determine whether or not the user can comfortably use the application free of stress based on the communication status measured in step S101. As a specific example, the controlling unit 11 may determine that the operation stream satisfies the predetermined condition if the measured communication status indicates that the number of errors in a communication between the computer apparatus 1 and the measurement device 3 is equal to or lower than a threshold. The threshold is stored in the storage device 12, for example.

(Step S104)

In next step S104, the controlling unit 11 functions as the request unit 113, and performs a display for requesting the user 5 to input and post a review of the application 121 through the touch panel display 14. In the present embodiment, in either a case where signs of improvement are found in the health condition of the user 5 in step S102, or a case where it is determined that the operation stream of the user 5 satisfies the predetermined condition in step S103, the processing in step S104 is performed.

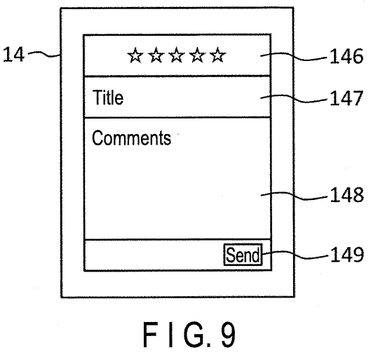

Herein, an example of a method of requesting a user to post a review will be described with reference to FIGS. 8 and 9. FIG. 8 shows an example of a screen for a request to post a review of the application 121, displayed on the touch panel display 14. FIG. 9 shows an example of a screen for accepting input of reviews.

First, the controlling unit 11 displays the screen shown in FIG. 8 on the touch panel display 14 in step S104 to request the user 5 to post a review of the application 121. The screen example shown in FIG. 8 includes, in addition to comments indicating a request to post a review, an “evaluate” button 141, an “evaluate later” button 142, and a “do not evaluate” button 143.

When the user taps the “evaluate later” button 142 or the “do not evaluate” button 143, this means the user is declining to post a review at this point in time. Thus, when the “evaluate later” button 142 or the “do not evaluate” button 143 is tapped, the controlling unit 11 dismisses the screen shown in FIG. 8 on the touch panel display 14. When the “evaluate later” button 142 is tapped, the controlling unit 11 dismisses the screen shown in FIG. 8, and then displays the same screen again on the touch panel display 14 after a certain period of time elapses. The above-mentioned certain period of time may be set as appropriate in accordance with an embodiment. In contrast, when the “do not evaluate” button 143 is tapped, the controlling unit 11 dismisses the screen of FIG. 8, and finishes the processing relating to the present operation example. In this case, after a certain period of time, which is longer than the certain period time for the case where the “evaluate later” button 142 is tapped, has elapsed, the controlling unit 11 may return to the processing in step S101, so that the screen of FIG. 8 is displayed once again on the touch panel display 14.

In contrast, when the user 5 taps the “evaluate” button 141, this means the user agrees to post a review at this point in time. For this reason, the controlling unit 11 displays the next screen shown in FIG. 9 on the touch panel display 14. The screen example of FIG. 9, a first area 146 for inputting evaluation points for the application 121 with a 5-point rating system, a second area 147 for inputting a title of a review, a third area 148 for inputting content (comments) of a review, and a send button 149. The user 5 operates the touch panel display 14, and inputs an evaluation point, title, and comments in the respective areas 146 through 148. After the input is completed, the input review can be posted by tapping the send button 149. In other words, when the send button 149 is tapped, the controlling unit 11 transmits the data input in the areas 146 through 148 to the server 7 as data for posting.

With such a method, the controlling unit 11 can request the posting of a review of the application 121, and accept the input of the review. However, the method of requesting the user to post a review is not limited to the above-described method. For example, in step S104, the controlling unit 11 may obtain a website for inputting a review from the server 7, display the obtained website on the touch panel display 14, and cause the user to input a review on the displayed website. When a request to post a review is completed, the controlling unit 11 may finish the processing relating to the present operation example.

[Features]

As described above, in the present embodiment, it is determined, in step S102, whether or not there are signs of improvement in the health condition of the user 5 through using the application 121 based on the collected biological data 123. Then, if it is determined that there are signs of improvement in the health condition of the user 5, the user is requested to post a review of the application 121 in step S104. In the present embodiment, it is thus possible to request the user 5 to write a review of the application 121 at a timing when the health condition of the user 5 is improved through the use of the application 121. Thus, as described above, it is possible to request the user, who highly values the application 121, to write a review at an appropriate timing, and thereby possible to collect more reviews with high ratings for the application 121.

In the present embodiment, the operation stream of the user 5 when the application 121 is used is specified in step S101, along with whether or not the specified operation stream satisfies the predetermined condition in step S103. Then, in the present embodiment, if it is determined that the operation stream of the user 5 satisfies the predetermined condition, the user 5 is requested to post a review of the application 121 in step S104. It is thereby possible, in the present embodiment, to request a user inferred to have a good impression of the application 121 from, for example, experience of using the application 121 comfortably and free of stress, based on an operation stream, to write a review of the application 121. For this reason, according to the present embodiment, it is expected that more reviews with high ratings can be collected for the application 121.

### 4. MODIFICATIONS

Although the embodiment of the present embodiment has been described in detail in the foregoing, the description is merely an example of the present embodiment in every respect. Various improvements and modifications can of course be made to the embodiment to the extent that no deviation is made from the scope of the present embodiment. The following modifications can be made for example. In the following, the same reference numerals are used for the same constituent elements of the foregoing embodiment, and redundant descriptions are omitted as appropriate. The following modifications can be combined as appropriate.

<4.1>

In the example of FIG. 1, the external server 7 has both the roles of providing the application (software) 121 and storing data of posted reviews. However, the configuration for providing the application 121 and the configuration for accumulating data of reviews are not limited to such an example. For example, the server that provides the application 121 may differ from the server storing the data of posted reviews. Furthermore, the application 121 may be provided through a storage medium, etc., not from an external server. A method of storing the data of posted reviews may be determined as appropriate in accordance with an embodiment, as long as the posted reviews can be utilized when providing and advertising the application 121.

<4.2>

In the foregoing embodiment, the biological data 123 measured by the measurement device 3 is stored in the computer apparatus 1. However, where the biological data 123 is stored is not limited to this example, and may be selected as appropriate in accordance with an embodiment. For example, the biological data 123 may be stored in a server apparatus, etc. on a cloud, similar to the external server 7. In this case, the server apparatus storing the biological data 123 may perform the processing in step S102 and step S104.

<4.3>

In the foregoing embodiment, whether or not the posting of a review of the application 121 is requested to a user 5 is based on two indices used in step S102 and step S103. However, it may not be necessary to use both indices. In other words, one of the determinations in step S102 and step S103 may be omitted. Types of the application 121 are not necessarily limited to the example described in the foregoing embodiment, and can be selected as appropriate in accordance with an embodiment. If step S102 is omitted and the communication status in step S103 is used, the application 121 is not limited to the above-described example as long as the application 121 is of a type that causes the computer apparatus 1 to communicate with a predetermined apparatus when predetermined data is collected. In this case, the type of the predetermined apparatus is not limited to the above-described measurement device 3 as long as it can communicate with the computer apparatus 1. A communication between the computer apparatus 1 and the predetermined apparatus is not limited to a wireless communication and may be a wired communication.

<4.4>

The computer apparatus 1 according to the foregoing embodiment may determine whether or not to request the user 5 to write a review of the application 121, using an index other than the above two indices.

For example, the controlling unit 11 may determine whether or not at least one of the following is equal to or greater than a predetermined threshold: frequency of using the application 121; a period of using the application 121; the number of measurement devices 3 being used; frequency of using the measurement device 3; or a period of using the measurement device 3. Then, for at least one of the frequency of using the application 121, the period of using the application 121, the number of measurement devices 3 being used, the frequency of using the measurement device 3, or the period of using the measurement device 3 is equal to or greater than a predetermined threshold, the controlling unit 11 may request the user 5 to post a review of the application 121. In this case, the predetermined threshold can be set as appropriate.

For example, the controlling unit 11 may determine whether or not an attribute (category, model, etc.) of the measurement device 3 being used satisfies a predetermined condition. Then, if the attribute of the measurement device 3 satisfies a predetermined condition, the controlling unit 11 may request the user 5 to post a review of the application 121. In this case, the predetermined condition can be set as appropriate. For example, the predetermined condition may be defined in such a manner that a request for posting a review is made if the measurement device 3 is a body weight and composition monitor.

<4.5>

In the foregoing embodiment, the application 121 is software that collects the biological data 123 from the measurement device 3 and manages the collected biological data 123. However, where the type of the application 121 is not limited to this example, and may be selected as appropriate in accordance with an embodiment. The evaluation condition defining whether or not the content is preferable to the user may be defined as appropriate in accordance with a type of the application.

For example, the application may be software that collects newspaper articles from a website through a network. In this case, collecting a number of newspaper articles matching a user's interest which exceeds a predetermined amount within a predetermined period of time through the use of the application may be defined as an evaluation condition. In other words, if newspaper articles matching a user's interest are collected in numbers which exceed a predetermined amount within a predetermined period of time through the use of the application, the controlling unit 11 may determine that the collected predetermined data matches an evaluation condition in step S102, and therefore execute step S104.

For example, the application may be software that collects entertainment information, such as that concerned with sports. In this case, a win for a team that the user supports may be defined as an evaluation condition. In other words, the controlling unit 11 may determine whether or not, in step S102, a team that the user supports wins a game based on the entertainment information collected through the use of the application. Then, if it is determined that the team that the user supports wins a game, the controlling unit 11 may perform step S104.

<4.6>

Furthermore, for example, the computer apparatus 1 according to the foregoing embodiment has the touch panel display 14 as an example of the “display device” of the present embodiment. However, the type of the display device is not limited to a touch panel display, and may be selected as appropriate in accordance with an embodiment. For example, the display device may be a well-known display, different from a touch-panel type. In this case, the computer apparatus 1 may be equipped with an input device, such as buttons, a keyboard, or a mouse, etc. to receive an input of a review from the user 5.

1. A non-transitory computer readable medium storing an evaluation request computer program which is executed by a computer, the computer comprising a display device, to provide the steps of:
a first step of communicating with a measurement device configured to perform a measurement on a body of an user to provide services, and operating an evaluation-target application that collects biological data indicating a result of the measurement as first data;
a second step of determining whether or not content of the first data collected through an operation of the evaluation-target application matches an evaluation condition defining whether or not the data is preferable to a user; and
a third step of causing the display device to display a screen for requesting the user to input a review of the evaluation-target application if content of the first data matches the evaluation condition.

2. The non-transitory computer readable medium according to claim 1, wherein
the evaluation-target application communicates with a measurement device configured to perform measurement of the body of the user, and collects biological data indicating a result of the measurement as the first data,
the evaluation condition is defined so as to determine that content of the first data is preferable to the user if there are signs of improvement in a health condition of the user,
in the second step, determining whether or not there are signs of improvement in the health condition of the user, based on the collected biological data,
in the third step, displaying a screen for requesting the user to input a review of the evaluation-target application if it is determined that there are signs of improvement in the health condition of the user.

3. The non-transitory computer readable medium according to claim 2, wherein
the biological data is related to at least one of blood pressure, body weight, body fat, visceral fat, physical age, skeletal muscle rate, number of steps, activity amount, blood glucose level, basal body temperature rhythm, or sleep condition, and
in the second step, determining whether or not there are signs of improvement in at least one of the blood pressure, the body weight, the body fat, the visceral fat, the physical age, the skeletal muscle rate, the number of steps, the activity amount, the blood glucose level, the basal body temperature rhythm, or the sleep condition.

4. The non-transitory computer readable medium according to claim 1, wherein the steps further comprising:
a fourth step of monitoring an operation stream of the user when the evaluation-target application is used,
a fifth step of determining whether or not the operation stream satisfies a first condition, and
a sixth step of causing the display device to display a screen for requesting the user to input a review of the evaluation-target application if it is determined that the operation stream satisfies the first condition.

5. The non-transitory computer readable medium according to claim 4, wherein
in the fourth step, the computer is caused to measure at least one of a length of time staying on an operation screen or an operation path as the operation stream,
in the fifth step, the computer is caused to determine whether or not the operation stream satisfies the first condition based on at least one of the length of time staying on the operation screen or the operation path.

6. The evaluation request program according to claim 4, wherein
the evaluation-target application causes the computer to communicate with a first apparatus when collecting the first data,
in the fourth step, measuring a status of a communication with the first apparatus as the operation stream, and
in the fifth step, determining whether or not the operation stream satisfies the first condition based on the communication status.

7. An evaluation request method of causing a computer comprising a display to execute:
a first step of communicating with a measurement device configured to perform a measurement on a body of an user to provide services, and operating an evaluation-target application that collects biological data indicating a result of the measurement as first data;
a second step of determining whether or not the content of the first data collected through an operation of the evaluation-target application matches an evaluation condition defining whether or not the data is preferable to a user; and
a third step of causing the display device to display a screen for requesting the user to input a review of the evaluation-target application if the content of the first data matches the evaluation condition.

8. The method according to claim 7, wherein
the evaluation-target application communicates with a measurement device configured to perform measurement of the body of the user, and collects biological data indicating a result of the measurement as the first data,
the evaluation condition is defined so as to determine that content of the first data is preferable to the user if there are signs of improvement in a health condition of the user,
in the second step, determining whether or not there are signs of improvement in the health condition of the user, based on the collected biological data, and
in the third step, displaying a screen for requesting the user to input a review of the evaluation-target application if it is determined that there are signs of improvement in the health condition of the user.

9. A computer apparatus comprising:
one or more processors;
a memory storing an evaluation-target application and a program in an executable manner for the one or more processors; and
a display device,
wherein the one or more processors execute the following in accordance with the program:
a first step of communicating with a measurement device configured to perform a measurement on a body of an user to provide services, and operating an evaluation-target application that collects biological data indicating a result of the measurement as first data;
a second step of determining whether or not content of the first data collected through an operation of the evaluation-target application matches an evaluation condition defining whether or not the data is preferable to a user; and
a third step of causing the display device to display a screen for requesting the user to input a review of the evaluation-target application if content of the first data matches the evaluation condition.

10. The apparatus according to claim 9, wherein
the evaluation-target application communicates with a measurement device configured to perform measurement of the body of the user, and collects biological data indicating a result of the measurement as the first data,
the evaluation condition is defined so as to determine that content of the first data is preferable to the user if there are signs of improvement in a health condition of the user,
in the second step, the one or more processors determine whether or not there are signs of improvement in the health condition of the user, based on the collected biological data, and
in the third step, the one or more processors display a screen for requesting the user to input a review of the evaluation-target application if it is determined that there are signs of improvement in the health condition of the user.
